(19) United States
(12) Patent Application Publication
(10) Pub. No.: US 2016/0136124 A1
(43) Pub. Date: May 19, 2016

(54) ADMINISTRATION OF A FOOD COMPOSITION PRODUCT

(71) Applicant: INDEVEX AB (PUBL), Vimmerby (SE)

(72) Inventors: Christer Franzén, Vimmerby (SE); Thomas Buchar, Jönköping (SE); Magnus Sederholm, Stockholm (SE)

(21) Appl. No.: 14/899,797

(22) PCT Filed: Jun. 13, 2014
(86) PCT No.: PCT/SE2014/050721

§ 371 Date: Dec. 18, 2015

(30) Foreign Application Priority Data
Jun. 19, 2013 (SE) 1350750-4

(51) Int. Cl. A61K 31/202; A61K 38/16; A61K 31/70; A61K 31/714; A61K 31/375; A61K 9/00; A61K 38/17; A61K 31/201

(52) U.S. Cl. CPC A61K 31/202; A61K 38/17; A61K 38/168; A61K 31/201; A61K 31/714; A61K 31/375; A61K 9/0095; A61K 31/70; A61K 9/0053; A23L 1/305; A23L 1/3008; A23L 1/308; A23V 2002/00

(57) ABSTRACT

Method for orally administrating a subject a food composition product, referred to as “Preload Meal,” before administering a main meal to the subject. The food composition product includes nutrients that stimulate the release of incretins in the subject prior to administration of the main meal, increasing satiety, lowering total energy intake from both the food composition product and the main meal, and contributing to a biological control of a post prandial blood sugar level and a post prandial insulin level in the subject.

### CROSS REFERENCE TO RELATED APPLICATIONS

This application is the U.S. National Phase Application under 35 U.S.C. §371 of International Application No. PCT/SE2014/050721, filed Jun. 13, 2014, designating the U.S. and published in English as WO 2014/204382 A1 on Dec. 24, 2014 which claims the benefit of Sweden Patent Application No. SE 1350750-4, filed Jun. 19, 2013. Any and all applications for which a foreign or a domestic priority is claimed is/are identified in the Application Data Sheet filed herewith and is/are hereby incorporated by reference in their entirety under 37 C.F.R. §1.57.

### TECHNICAL FIELD

The invention relates to a method of administrating of a nutritional composition, the therapeutic diet intervention concept “Preload” including a food composition product “Preload Meal” to be taken before main meals.

### BACKGROUND

65% of the world's populations live in countries where overweight and obesity kills more people than underweight. Worldwide obesity has nearly doubled during the last three decades, now approx. 1.5 billion adults (20 years and older), were overweight defined as BMI>25. Of these over 200 million men and 300 million women are obese, defined as BMI>30. An even more alarming fact is the growing obesity among children; more than 40 million children under the age of five were overweight in 2011. Once considered a high-income country problem, overweight and obesity are now on the rise in low- and middle-income countries, particularly in urban settings.

Similar alarming increasing development of diabetes typ 2 is ongoing in a big number of areas in the world.

Obesity is regarded as major risk factor for several serious diseases such as cardiovascular disorders, hypertension, diabetes typ-2, cancer and osteoarthritis.

Conditions as described above can be prevented through lifestyle modification, diet control, and control of overweight and obesity. Education of the populace is still key to the control of this emerging epidemic. Novel drugs are being developed, yet no definite cure is available in sight for obesity and its alarming consequences like CVD and diabetes type 2. On the other hand life style and diet interventions have scientifically strong support in preventing the onset of e.g. type 2 diabetes as well as to improve the manifest disease.

A common way of dealing with obesity is weight loss through diets. However, diets may lead to poor nutritional intake or even nutritional deficiencies.

There is thus a need for an improved approach towards controlling metabolism and stabilizing blood sugar while still providing essential nutrition in order to fight overweight, obesity and the increased medical risks related to unhealthy diets, diabetes type 2, cardiovascular diseases and cancers. The majority of the popular low calorie diets have failed to show sustainably weight loss. Recently the Diogenes study, supported by the EU Commission, has shown that the only long term sustainable nutrient combination to keep weight stable after weight reduction is combination of high protein content with low GI (N Engl J Med 363;22, 2010)

### SUMMARY OF THE INVENTION

The object of the present invention is to provide an inventive new method for increasing satiety and controlling blood sugar levels in a subject after the administration of a meal to said subject, such that previously mentioned problems are avoided. The method is called “Preload” and the food composition product used “Preload Meal”, and refers to a load of nutrients 15-45 minutes before a meal in order to reduce postprandial levels of blood sugar.

### BRIEF DESCRIPTION OF THE DRAWINGS

FIG. 1a shows a line graph of the average weight loss in subjects participating in an embodiment of a weight loss program according to an embodiment of the present disclosure.

FIG. 1b shows a bar graph of reduced sugar cravings according to VAS scale measurements before and after an intervention according to an embodiment of the present disclosure.

### DETAILED DESCRIPTION

The invention relates to a method for orally administrating a subject a food composition product including 50 to 110 kcal “Preload meal” before administrating a main meal, i.e. breakfast, lunch or dinner, to said subject and said food composition product to include nutrients, such that release of incretins in said subject is stimulated prior to said main meal, said food composition product comprising of at least two different proteins deriving from both animal and vegetable sources, furthermore Omega 3 and 6 fatty acids and slowly digested carbohydrates including dietary fibers, giving said composition a low glycemic load (GL<10), thereby increasing satiety of subject and lowering total energy intake including both food composition product and main meal; the administration of said food composition product, Preload Meal, contributing to a biological control of the post prandial blood sugar and insulin levels.

The method comprises the steps of: preparing a composition as a food product i.e. Preload Meal, the composition comprising three proteins sources: pea protein, whey protein, egg albumen, omega 3 rich eggs, apple and sugar root fibre, and orally administrating said food product of said composition to a subject prior to administrating a meal to said subject such that release of incretins in said subject is simulated prior to said meal administration.

The invention also concerns preparing a composition as a food product, which given as a preload, increase satiety and stabilize the blood sugar level. The food composition product comprising at least two but even more preferably four proteins sources: leguminous plants preferably pea protein, whey protein, egg albumen and casein protein, furthermore omega 3 and 6 fatty acids, rosaceous plants preferably apple and rose hip, and also fibers preferably from sugar beet root fibre and orally administrating said Preload Meal including said food composition product to a subject prior to administrating a main meal to said subject i.e. breakfast, lunch or dinner, such that release of the gastrointestinal hormones incretins (see below) in said subject is stimulated prior to said main meal administration.

Proteins have multiple points of action in diabetes and weight management; slows the emptying of the stomach contents (takes longer for carbohydrates to be absorbed), stimulate faster release of insulin (rapid uptake of glucose into tissues), stimulates the synthesis of muscle tissue and impact the degree of saturation, etc. through neurotransmitters and neuropeptides.

Pea protein are among the lowest glycemic index (GI) foods and have been recommended in national diabetes mellitus (DM) guidelines. Incorporation of legumes as part of a low-GI diet improves both glycemic control and reduced calculated CHD risk score in type 2 DM in a recent study (Arch Intern Med. 2012 Nov 26;172(21):1653-60). It also has been demonstrated that intact pea proteins stimulates CCK and GLP-1 release from human duodenal tissue to a greater extent than egg and codfish protein. Unlike other common protein sources such as milk, soy, or wheat proteins, pea protein has a very low allergenic potential, which makes this protein suitable for dietary interventions (PLoS One. 2011;6(9):e24878). In another study of preload, food intake was significantly lower only after casein and pea protein compared to water control (P=0.02; 0.04 respectively) (Nutrition Journal 2011, 10:139)

The combination of proteins and omega fatty acids in the food product strengthen the effect. In a recent review of omega fatty acids it was concluded that there is a considerable body of evidence from animal studies indicating that supplementing the diet with LC n-3 PUFA can attenuate weight gain and reduce body fat, in particular epididymal (visceral) fat. Similarly, in human studies there is a growing body of evidence indicating that increasing the intake of LC n-3 PUFA by 0.3-3.0 g/day can reduce body weight and body fat in overweight and obese individuals (Nutrients 2010, 2, 1212-1230). It also has been shown that long-term enteral provision of LCn-3PUFA confers a higher sensitivity to insulin-regulated amino acid and glucose disposal and that these responses probably occur, in part, in skeletal muscle. In a fed steady-state, a more sensitive insulin signalling promotes initiation of protein synthesis with concurrent reduction in whole-body amino acid oxidation, increasing the net availability of amino acids to support anabolism, of paramount importance during weight control (shift from fat body mass to lean body mass).

The inclusion of fibers in the food composition product further promotes satiety at following meals. In a controlled study the fiber supplement significantly reduced hunger feelings and promoted satiety during a period of caloric reduction (Nutr Diabetes. (2011);1:e22). Most clinicians in the field of obesity medicine agree that dietary intervention for long-term weight management should include volumetric, low-calorie-density, low GI diets supplemented with moderate amounts of protein. Increasing the intake of fiber should play a central role in this regard. Supplementation of the diet with functional fibers may significantly promote satiety and reduce cardiometabolic risk factors according to a recent review (Curr Obes Rep (2012) 1:59-67).

The incretins are important gut mediators of insulin release and plasma glucose levels. A food product of the composition administrated prior to a major meal stimulates the release of the incretins GIP and GLP-1. The neuroendocrine response reaches the brain through the systemic route and through afferent neurons. Administration of certain nutrients before a meal, Preload, activates the normal incretin response before the ordinary meal, i.e it has a priming effect on the brain. When the meal is ingested the GIP/GLP-1 already has stimulated the insulin release, promoted satiety and slowed gastric emptying (the priming effect), which in turn reduce meal size and attenuates plasma glucose response.

Preload of proteins/nutrients 15-45 minutes before a meal to the subject is beneficial because the maximum insulin response occurs 30 minutes after ingestion of proteins together with carbohydrates. Thus, initiation of administration of a food product of the composition is preferably made 15-45 minutes prior to the administration of a meal, more preferably 25-35 minutes prior.

The Preload Meal composition administered as a preload 15-45 minutes before major meals during a longer period (>3 months) significantly reduced body weight, BMI and fat %. The food composition product also has positive effects on blood lipids, liver enzymes, insulin levels and HbA1C, and improved life quality parameters on VAS-scales.

Besides proteins nutritional role as a source of “building blocks” to the tissues, dietary proteins affect many vital functions such as glucose and lipid metabolism, blood pressure, bone metabolism and the immune system. Proteins can also function as a “fuel”, with approximately the same energy content as carbohydrates. Proteins affect metabolism throughout the entire nutrient uptake, from the oral cavity to the colon. The proteins have several regulatory functions in the gastrointestinal system, including the regulation of food intake. The interaction with signal receptors release hormones that influence gastric emptying and transport of food through the GI system and absorption of nutrients. The proteins also induce nerve signals to the brain (eg, the degree of satiation) through gut receptors and by affecting the intestinal microflora. The interaction between proteins' degradation products (peptides, amino acids) and the endocrine systems also affects digestion.

A dose, i.e. a food product, with a minimum of to 5 grams protein when administrated prior to the administration of a meal is sufficient to affect insulin and glycemic response after the meal.

The four different preferred proteins sources that may be included in a Preload Meal i.e; yellow pea, chicken egg, whey and casein, have different kinetics and effects on the plasma glucose levels, incretins and satiety system. The ingredient omega 3 fatty acid can potentiate the effect of the proteins regarding the above effects.

The method may also comprise the step of preparing the food product of the composition by mixing the composition with a drinkable liquid. The composition is preferably prepared in dry powder form such that a food product may be prepared as a shake. A serving of the food composition product, prior to a meal, should be approximately 70 kcal. Therefore, the weight of the powder to be mixed in the shake is determined based on calories. The food product may also be prepared as a ready-to-drink liquid comprising a dose of the food composition product.

A food product of the composition for mixing with a drinkable liquid according to the invention is distinguished primarily by the fact that the composition comprises all the essential food components readily accessible, wherein the relative amounts of the components fat, carbohydrates, proteins, vitamins and minerals of said composition are chosen such that an intake of said composition provides the consumer with a stable blood glucose level.

Said fat, carbohydrates, proteins, vitamins and minerals are derived from a diversity in food groups including fruit, vegetable, plant, dairy, egg and other protein sources, and colloidal water sources, resulting in a physiologically balanced composition.

The “glycemic index” (GI) is a measure of the degree to which the concentration of glucose in the blood rises after consumption of certain foodstuffs. A low glycemic index (GI) here refers to foodstuffs with a GI-value between 0-55. The glycemic index (GI) may be calculated using two different references, that is to say either the reference white bread or the reference glucose. In the measurements made on the food composition product according to the invention, the reference glucose was utilised. In order to estimate the overall glycemic effect of a meal, the concept of “glycemic load” (GL) (GI×dietary carbohydrate content) has been introduced. As the GI compares corresponding amounts of carbohydrates, providing a measure of carbohydrate quantity, but not quality, the GL-value provides the glycemic effect of realistic portion sizes of different foods. The GL-value is similar to the GI-value, a measure of the rise of the blood glucose and the subsequent secretion of insulin in the blood stream, but including the aspect of the amount of carbohydrates available in a portion of food (see Foster-Powell K., Holt S H., Brand-Miller J C., Am. J. C/in. Nutr. 76:5-56, 2002).

Such a food composition product formulation is carefully chosen to support the body with essential nutrition. Evidence based medicine support the rationale behind the formula. The food composition product contains a protein, fat and complex carbohydrate combination, based on pure natural sources. It also contains slow carbohydrates and dietary fibers that together with the proteins and fats add up to a low glycemic index of below 40 and more preferably below 30%. With the comparingly low carbohydrate content this adds up into a lo glycemic load (GL) for the Preload Meal of not more than 10 and preferably below 5 which provides a low and stable post prandial blood sugar and equally insulin levels, and in combination with the protein and fat load also a high satiety.

According to one preferred embodiment of the invention, the carbohydrates are derived from leguminous plants, preferably yellow peas, and rosaceous plants, preferably apples and rose hips, wherein said leguminous plants and rosaceous plants contribute in giving the food composition product its advantageously low GIvalue. However, carbohydrates may, of course within the scope of the invention, be chosen from other plants having a low glycemic index. Apples and rose hips may furthermore be substituted with pears, peaches, plums, or the like, all of which being rosaceous plants with a low glycemic index. In a further preferred embodiment of the invention, at least 15% of the overall content of the food composition product is derived from yellow peas, at least 10% from apples or other rosaceous plants. The content of the mentioned ingredients preferably does not exceed 30% for yellow peas and 25% for apples or other rosaceous plants so as to maintain the important balance between carbohydrates, proteins and fat.

In addition, said food composition product is also proven to have a satiating effect for a longer period of time after a consumed meal, which effect is of great importance so as to keep feelings of hunger away. As has been disclosed in a previous study (Holt S H., Miller J C., Petocz., Farmakalidis E., Eur. J. Clin. Nutr. 49(9):675-690, 1995), different foods differ greatly in their satiating capacities. The food composition product according to the invention provides said satiating effect due to a number of reasons mentioned in the study above. The low degree of fat in the food composition product is proven advantageous, as fatty foods are shown to be less satisfying, as well as the inclusion of foodstuffs such as eggs and apples, both of which having a high satiety index, as defined in said article. Furthermore, the food composition product according to the invention has a balanced ratio between certain important components such as the contents of protein, fibre, and water. Said components are shown in the above mentioned study to have a positive effect on said satiety.

The food composition product may be balanced to have a GL-value below 10, preferably below 5. As earlier mentioned, carbohydrates are metabolised into glucose by the digestive system of the human body. Until the mid-eighties, it was generally considered that the size of the carbohydrates was of primary importance regarding the blood glucose response. Today, it is known that the same carbohydrate gives rise to different blood glucose responses, due to the form in which it is included in the foodstuffs (see e.g. Björck I., Liljeberg H., Granfeldt Y, \dot{A}kerberg A., Scand. J. Nutr./Näringsforskning Vol. 40:38-42, 1996). The “glycemic index” (GI) has replaced the terminology of “fast” and “slow” carbohydrates which were related to the size of the sugars included. High GI-values indicate a rapid increase in blood glucose, and low GI-values indicate a delayed absorption rate for the glucose; i.e. carbohydrates with low GI-values are more slowly digested and absorbed. As the level of blood glucose is kept at a more even level, feelings of hunger are kept away for a prolonged period also avoiding unnecessary and unhealthy cravings for foodstuffs rich in carbohydrates. Thus, said problems are avoided with the food composition product having said GL-value, which GL-value is a product of a low GI-value and the amount of available carbohydrates. However, in addition to the food composition product having a low GL-value, the satiating effect of said product is of equal importance in order to avoid feelings of hunger.

However, even though carbohydrates from different sources may be associated with individual GI-values, the GI-value of each of the carbohydrates put together does not determine the GI of the foodstuffs. The GI is influenced by a number of factors, for example, the biochemical structure of the carbohydrate, a high amylose/amylopectin ratio, a high degree of native starch, presence of anti-nutritional substances with the ability of inhibiting amylose, and the co-ingestion of fat, fibre and protein. A problem thus occurs when trying to compose a meal which is to provide the consumer with a low glycemic index for the overall food composition product. Due to the contribution of a number of factors to the glycemic index, a meal properly balanced so as to avoid fluctuations in the blood glucose level may be difficult to compose.

Furthermore, the choice of carbohydrate source is, as also mentioned, another important factor in obtaining a low GL food composition product. Accordingly, carbohydrates having low GI-values are chosen. According to one embodiment of the invention the food composition product has a content comprising yellow peas, apples, and rose hips as described above, all of which comprise carbohydrates with low GI-values. The present inventors have found that when foodstuffs with an average GI-value, that is between 60-90, are included, the overall food composition product obtains a significantly higher GL-value when compared to a food composition product comprising only carbohydrates with a low GI-value.

According to a further embodiment of the invention, the carbohydrates, when decomposed provide the human body system with simple sugars from the group consisting of glucose, saccharose, fructose, maltose and lactose.

However, it is also possible to alter the contents of the different types of sugar within the scope of the invention. In one embodiment according to the invention the food composition product is low lactose or completely free from lactose in order to meet the demands for such products.

According to one embodiment of the invention, the food composition product comprises physiological doses of dietary fibres, i.e. between 3-15 g of 100 g respectively 1.5-7.5 g of 100 kcal of a Prelaod Meal preferably about 5-10 g per 100 g resp 2.5-5 g per 100 kcal and most preferably 6 g per serving in a non liquid Preload Meal. The GL of a meal is affected by the content of fibres, as a high degree of fibres helps to lower the glycemic index. Fibres are not digested, but are however necessary in order for the bowels to function correctly. The content of fibres in the food composition product according to the invention is advantageous in improving motility (bowel movements) and thus enhancing the consumer's ability to process food efficiently. In addition, the fibres help the elimination of toxins in the cells and furthermore is recognized to reduce the cholesterol content in the blood system.

In a preferred embodiment according to the invention the fat of the Preload Meal comprises the essential fatty acids with an omega-3 to omega-6 fatty acid ratio of 1:1 to 1:4.0, more preferably 1:1.2 to 1:3.0, and most preferably 1:1.5 to 1:2.5. A correct balance is significant in order to maintain normal cellular and other functions. As a typical western diet consists of far more omega-6 fatty acids than omega-3 fatty acids, many meals are lacking the essential amount of the desired fatty acids and have moreover an inadequate balance of said fatty acids. The fat of the composition is further composed to meet the needs for saturated, and mono-, di-, tri- and polyunsaturated fatty acids. The fat preferably comprises preferably 20-40% saturated fatty acids, preferably 20-40% monounsaturated fatty acids, and preferably 20-40% di- and tri-unsaturated fatty acids. The balance between the three main groups of fatty acids is thus optimised. The content of fat in a properly balanced meal is essential; fats contain important fat soluble vitamins and essential fatty acids which cannot be produced by the human body. The fatty acids are preferably chosen from the group consisting of myristic acid, palmitic acid, palmitoleic acid, heptadecanoic acid, stearic acid, oleic acid, linoleic acid, alfa-linolenic acid, arachidic acid, eicosadienoic acid, behenic acid, docosahexaenoic acid (DHA) and eicosapentaenoic acid (EPA). However, the invention is not restricted to the fatty acids mentioned herein. Other fatty acids which are within the scope of the invention may also be chosen. In addition, the levels of docosahexaenoic acid (DHA) and eicosapentaenoic acid (EPA) are optimised and constitute between 1.9-2.3%, preferably about 2.1% of the total fat. Cholesterol is also present in amounts which meet the body's needs.

The composition is furthermore an excellent source of protein, thus containing all 20 amino acids including the 8 for adult by WHO recognized essential amino acids in physiological doses. The composition further comprise vitamins comparingly high amounts of vitamin B12 and C.

The food composition product mixed with drinkable liquid preferably has a pH-range of 5.5 to 6.5 and acts like a buffer.

In order to meet the demands of people with gluten intolerance, the food composition product, Preload Meal, is preferably gluten free.

Furthermore, said food composition product may also be provided in the form of a bar, thus a Preload Meal food product of the composition is easy to administrate and consume. The bar may be produced by any method known in the art.

The nutritive substances of the food composition product according to the invention are derived from both animal and vegetable sources, thereby providing all the amino acids, including the essential amino acids, fatty acids, and slow-working carbohydrates in a proper balance, all of which contribute to a low glycemic index. No artificial sweeteners, preservatives or thickening agents are added to the food composition product.

All ingredients are mechanically treated (heat, air, steam, pressure) to form a powder and are free from chemicals and are GMO free.

Said food composition product has also proved to provide a stable blood glucose level, to prevent insulin spikes. In addition, basal insulin levels are lowered in all people, and long acting (basal insulin level) and short acting (insulin spike) insulin requirements are significantly reduced with insulin dependent diabetics.

In an embodiment of the invention the food composition product the components includes a protein content of more than 20% calculated from the food composition products energy content; and thus according to European/EFSA regulations classified as a “protein rich” food composition.

The invention relates to a method, as disclosed herein, for orally administrating a subject a food composition product wherein the method comprising administration of said food composition product contributing to a biological control of the post prandial blood sugar and insulin levels for non-medical weight management.

The invention further relates to the food composition product as outlines herein for use in treatment of diabetes and obesity by means of the method for orally administrating a subject the food composition product as outlined herein.

### EXAMPLE 1

Study on the Effect of Administrating the Composition Prior to a Meal

In a study performed at Lund University a cohort of obese subjects was administrated a dose of the composition, as a water soluble dry powder/shake, 2 to 3 times a day as preload before major meals. Parameters linked to obesity and diabetes type 2 was documented. 12 subjects with a BMI>30, 8 women and 4 men with a mean age of 43 years, ranging between 25 and 58 years, participated in a special program for weight reduction. The program was constituted by intake of the food composition product and access to a nutritionist by whom the participants was given nutritional advice as well as guidance to a healthier life style. The included subjects were non-smokers and, besides the overweight, without any known diseases. Medical examination was performed at the start of the program and at 3, 6, 12 and 24 months for blood sampling, answering questions, and to get advice. DEXA, blood pressure, spirometry, ECG, body weight, height, circumference of waist and hip were recorded.

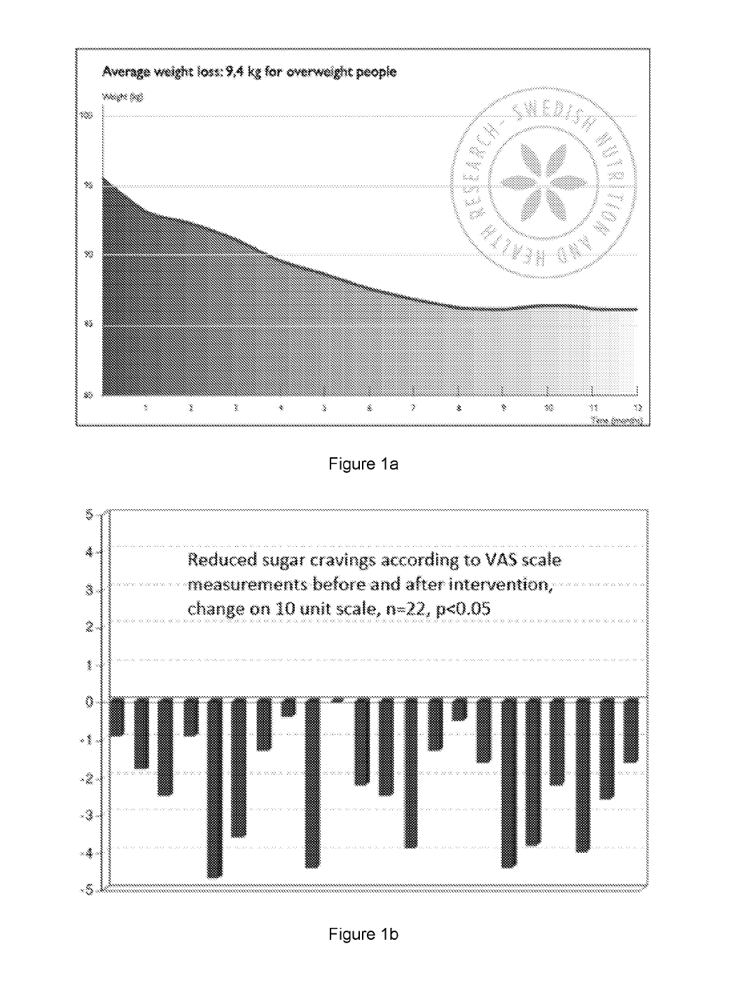

The mean body weight at the end of each period of the 1 year and 2 year groups decreased with 9.4 kg and 13.6 kg, respectively compared to baseline. FIG. 1a shows the average weight loss over the first 12 months. The BMI index decreased in analogy with the decrease in body weight. The waist and bottom circumferences also showed substantial decreases parallel to the body weight and BMI-index curves. In the VAS scale measurements, the desire for sweet and candies markedly reduced in the 1 and 2 year groups in all subjects. The level of energy and amount of exercise increased markedly for all subjects in both groups. All subjects in the 2 year group reported an increased feeling of general well-being.

In conclusion; the food composition product administered as a Preload Meal 30 minutes before major meals significantly reduced body weight, BMI and fat %. The composition also had positive effects on blood lipids, liver enzymes, insulin levels and HbA1C, and improved life quality parameters on VAS-scales.

As seen in FIG. 1a the subjects participating in the program had a substantial weight loss during the first year. During the second year the weight loss continued (2 year group). The BMI- values decreased from 33.4 to 30.5 in the 1 year group and from 34.9 to 30.3 in the 2 year group which is close to a WHO classification as overweight instead of being classified as obese. The circumference of waist and hip decreased in analogy with the weight. In parallel there was a reduction of the subjects' blood pressure.

The first three months after inclusion in the program the insulin levels reduced significantly and then levelled out at values between 9 and 10 mU/L. As long as a subject had a high insulin level it was impossible for him/her to lose weight, but when the insulin was reduced he/she started to lose weight. HbA1c reduced significantly the first three months and then there was an increase so that the values leveled out around 4.4%, which is normal.

FIG. 1b shows the reduced sugar cravings according to VAS scale measurements before and after intervention, change on 10 unit scale, n=22, p<0.05.

1.-14. (canceled)

15. A method for orally administering a food composition product to a subject, wherein:
the food composition product, referred to as a “Preload Meal,” comprises about 50 to about 110 kcal;
the food composition product is orally administered before orally administering a main meal to the subject;
the food composition product comprises nutrients that stimulate the release of incretins in the subject prior to orally administering the main meal, wherein the nutrients comprise:
a protein component, comprising at least two different protein sources derived from both an animal source and a vegetable source,
a fat component, comprising Omega 3 and Omega 6 essential fatty acids, and
a carbohydrate component, comprising slowly digested carbohydrates including dietary fibers; and
the food composition product has a glycemic load of less than 10;
wherein orally administering the food composition product increases satiety in the subject and lowers a total energy intake from both the food composition product and the main meal, and contributes to a biological control of a post prandial blood sugar level and a post prandial insulin level.

16. The method according to claim 15, wherein the main meal is at last one of breakfast, lunch or dinner.

17. The method according to claim 15, wherein orally administering the food composition product is initiated about 15 minutes to about 45 minutes prior to orally administering the main meal.

18. The method according to claim 15, wherein the food composition product comprises at least 5 grams and not more than 20 grams of protein.

19. The method according to claim 15, wherein at least one protein source comprises a leguminous plant.

20. The method according to claim 19, wherein the leguminous plant is yellow pea.

21. The method according to claim 15, wherein at least one additional protein source is either selected from the group consisting of whey, casein, chicken whole egg and egg albumin, or selected from a soy source.

22. The method according to claim 15, wherein at least 15% of an overall content of the food composition product is derived from yellow peas.

23. The method according to claim 15, wherein the carbohydrate component is at least one of a rosaceous plant and sugar beet fibers.

24. The method according to claim 15, wherein at least 10% of an overall content of the food composition product is derived from apples.

25. The method according to claim 15, wherein the fat component comprises a ratio of Omega 3 fatty acid to Omega 6 fatty acid of about 1:0.8 to about 1:4.

26. The method according to claim 15, wherein the fat component comprises a ratio of Omega 3 fatty acid to Omega 6 fatty acid of about 1:1.5 to about 1:2.5.

27. The method according to claim 15, wherein the fat component comprises a ratio of Omega 3 fatty acid to Omega 6 fatty acid of about 1:2.

28. The method according to claim 15, wherein the fat component additionally comprises about 20% to about 40% saturated fatty acids, about 20% to about 40% monounsaturated fatty acids, and about 20% to about 40% di- and tri-unsaturated fatty acids.

29. The method according to claim 15, wherein the food composition product comprises dietary fibers, wherein the dietary fibers comprise about 3 g to about 15 g per 100 g of the food composition product and respectively providing about 1.5 g to about 7.5 g per 100 kcal of the food composition product.

30. The method according to claim 15, wherein the food composition product comprises dietary fibers, wherein the dietary fibers comprise about 5 g to about 10 g per about 100 g of the food composition product and respectively providing about 2.5 g to about 5 g per about 100 kcal of the food composition product.

31. The method according to claim 15, wherein the food composition product comprises dietary fibers, wherein the dietary fibers comprise about 6 g per about 100 g of the food composition product and respectively providing about 3 g per about 100 kcal of the food composition product.

32. The method according to claim 15, wherein the food composition product further comprises all 20 amino acids including the 8 essential amino acids.

33. The method according to claim 15, wherein the food composition product further comprises vitamin B, vitamin C and sodium.

34. The method according to claim 15, wherein the method further comprises preparing the food composition product by mixing the nutrients with a drinkable liquid.

35. The method according to claim 1, wherein the food composition product is a buffer with a pH range of about 5.5 to about 6.5.

36. The method according to claim 1, wherein the food composition product comprises neither artificial preservatives nor thickening agents.

37. A food product composition, comprising:
a carbohydrate component, comprising carbohydrates with a glycemic index value of less than 55; and
a fat component, comprising a ratio of Omega 3 fatty acid to Omega 6 fatty acid of about 1:1,
wherein the food product composition has a balanced composition to provide a glycemic load value of less than 10 to an overall meal; and
wherein a relative amount of a component in the food composition product is such that an intake of the food composition product provides a stable post prandial blood sugar level and a stable post prandial insulin level.

38. The food composition product according to claim 37, wherein the composition, referred to as a “Preload Meal,” is orally administered to a subject about 15 minutes to about 45 minutes prior to an intake of a main meal by the subject.

39. The food composition product according to claim 37, wherein the fat component has a ratio of Omega 3 fatty acid to Omega 6 fatty acid of about 1:4.

40. The food composition product according to claim 37, wherein the food composition product is for treating at least one of diabetes type 2, overweight or obesity.
